(19) United States
(12) Patent Application Publication
(10) Pub. No.: US 2022/0214338 A1
(43) Pub. Date: Jul. 7, 2022

(54) SARS-CoV-2 (COVID-19) NEUTRALIZING ANTIBODY TEST KIT AND TEST METHOD USING THE SAME

(71) Applicant: VIRALQ BIOMEDICAL CO., LTD., TAIPEI CITY (TW)

(72) Inventors: TSU MIN HUANG, TAIPEI CITY (TW); ERR CHENG CHAN, TAIPEI CITY (TW)

(21) Appl. No.: 17/193,349

(22) Filed: Mar. 5, 2021

(30) Foreign Application Priority Data
Jan. 4, 2021 (TW) 110100162

(51) Int. Cl. G01N 33/543; G01N 33/52; G01N 21/78

(52) U.S. Cl. CPC G01N 33/54386; G01N 21/78; G01N 33/523

(57) ABSTRACT

The invention provides a test kit and a test method using the same. The test kit utilized for detecting the neutralizing antibody against COVID-19 has high sensitivity and specificity. The test kit especially comprises antigen-complex that essentially consists of a receptor binding domain (RBD) located in COVID-19 spike glycoprotein, two N-terminal domains located in COVID-19 spike glycoprotein and a domain between the RBD and the N-terminal domain (NTD). The test kit may quantitatively determine the titer of the neutralizing antibody against COVID-19.

This application claims priority of Application No. 110100162 filed in Taiwan on 4 Jan. 2021 under 35 U.S.C. § 119; the entire contents of all of which are hereby incorporated by reference.

### BACKGROUND OF THE INVENTION

### Field of the Invention

The present invention relates to a test kit and test method thereof, and in particular to the detection of SARS-CoV-2 (COVID-19) neutralizing antibody and provide antibody titer quantitative results.

### Description of the Prior Art

Severe acute respiratory syndrome coronavirus 2 (SARS-CoV-2 or COVID-19) is an enveloped non-segmented positive-sense RNA virus. It is the cause of coronavirus disease 2019, which is contagious in humans. COVID-19 has several structural proteins including spike glycoprotein (S), envelope (E), membrane (M) and nucleocapsid (N). The spike glycoprotein (S) contains a receptor binding domain (RBD), which is responsible for recognizing the cell surface receptor, angiotensin converting enzyme-2 (ACE2). It is found that the RBD of COVID-19 S protein strongly interacts with the human ACE2 receptor leading to endocytosis into the host cells and viral replication.

Infection with the COVID-19 initiates an immune response, which includes the production of antibodies in the blood. Generally, antibodies can be divided into two categories according to their binding properties to viruses: the first category is binding antibody, and the second category is neutralizing antibody. The binding antibody can bind to a specific antigen on the virus, but it may not necessarily inhibit the virus from infecting cells. In some cases, it may also cause antibody-dependent enhancement (ADE), which in turn strengthens the virus infection; therefore, the binding antibody cannot reflect real protection. The other type of antibody is neutralizing antibody, it remains in the circulatory system for months to years after infection and will bind quickly and strongly to the pathogen to block cellular infiltration and replication. The neutralizing antibody provides real protection against future infections from viruses.

Laboratory tests are not routinely performed to diagnose coronavirus infections other than SARS. The conventional method of choice for coronaviruses, including SARS-CoV, is detection of the viral RNA genome in respiratory and stool samples by RT-PCR, which takes hours. Virus isolation of the coronaviruses is difficult and for SARS-CoV requires stringent biosafety level 3 (BSL-3) conditions. Testing of samples suspected of containing SARS-CoV must be performed with appropriate BSL-2 precautions, attainable in many virology laboratories. When a large number of samples need to be tested, it will cause difficulties in actual implementation.

What is more, interpretation of test results of conventional immune test devices usually presents as follows: positive when the color in the test line region is darker than or equal to the control line region; negative when the color in the test line region is lighter than the control line region or no color appears in the test line region. Though conventional immune test devices can provide positive or negative results, they cannot quantitatively determine the titer of the neutralizing antibody.

### SUMMARY OF THE INVENTION

There is still a lot for improvement for those conventional coronavirus laboratory diagnostic methods and immune test devices. For example, virus isolation is not easy, RT-PCR is time-consuming, and detection of the neutralizing antibody for coronavirus requires a stringent facility to perform. In addition, conventional immune test devices cannot quantitatively determine the titer of the neutralizing antibody. The emerging COVID-19 has ravaged the world since 2020; therefore, to develop a rapid COVID-19 neutralizing antibody test kit and test method thereof with high sensitivity and specificity, more importantly, with quantitative analysis effect is necessary and urgent.

The test kit of the present invention comprises color particle, antigen-complex, testing reagent and controlling reagent, wherein the antigen-complex is a receptor binding domain (RBD) located in COVID-19 spike glycoprotein, two N-terminal domains located in COVID-19 spike glycoprotein, a domain between the RBD and the N-terminal domain, or a combination thereof.

To evaluate the sensitivity and specificity of the present invention, COVID-19 positive sera were bought from Access Biologicals LLC. 35 COVID-19 positive serum specimens were tested without any neutralizing antibody false negative result found, which indicated the sensitivity of the present invention is 100%. 85 COVID-19 positive serum specimens and high-risk groups COVID-19 negative serum specimens were tested with 1 neutralizing antibody false positive result found, which indicated the specificity of the present invention is 98.8%.

At present, there is no approved test kit related to the detection of SARS-CoV-2 (COVID-19) neutralizing antibody on the market. Compared with the traditional viral neutralization assay, the present invention can reach the same detection limit.

The invention provides a rapid quantitative assay for the titer assessment of neutralizing antibodies against COVID-19. Human plasma, serum, whole blood, urine and saliva can be used as specimen, and the test result is for (1) evaluating the convalescent plasma whether can be used to treat the infected patients fighting COVID-19, (2) assessing vaccine efficacy of COVID-19 vaccinated individuals whether to induce neutralizing antibodies responses, and (3) referring the levels of neutralizing antibodies in individuals recovered from COVID-19 or vaccine recipients to provide “immunity passport” whether they can go back in social gatherings without risk of infecting others.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the features and advantages of the invention. In the drawings:

FIG. 1 is a schematic drawing illustrating the test kit of the present invention;

FIG. 2 is a schematic drawing illustrating the interpretation of test results after utilizing the test kit of the present invention;

FIG. 3 is an actual test result of a representative lateral flow format (strip) of the present invention;

FIG. 4 is an antibody titer quantitative diagram of the viral neutralization assay and the test kit of the present invention. The X axis indicates titer, and the Y axis indicates ratio of the test line region (T) and the control line region (C) (T/C ratio);

FIG. 5 a flow chart illustrating the method for detecting neutralizing antibody against COVID-19 with the usage of the test kit of the present invention.

### DESCRIPTION OF THE PREFERRED EMBODIMENTS

Preparation of the SARS-CoV-2 (COVID-19) Neutralizing Antibody Test Kit

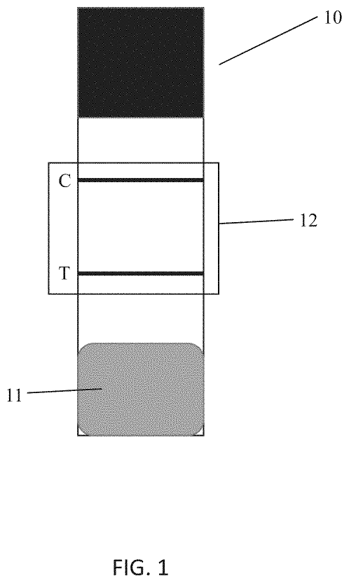

The present invention provides a preferred embodiment, a lateral flow format 10. Please refer to FIG. 1. The lateral flow format 10 comprises an absorbent pat 11 and a cellulose membrane 12, wherein a color particle and an antigen-complex are immobilized at the absorbent pat and the color particle are conjugated with the antigen-complex. The antigen-complex can be a receptor binding domain (RBD) located in COVID-19 spike glycoprotein, two N-terminal domains located in COVID-19 spike glycoprotein, a domain between the RBD and the N-terminal domain, or a combination thereof. The color particle comprises a colloid gold, a latex particle and a silver particle. The cellulose membrane 12 further comprises a test line region (T) printed with a testing reagent and a control line region (C) printed with a controlling reagent. The testing reagent is goat anti-human IgG antibody, and the controlling reagent is rabbit anti-RBD antibody.

The preferred embodiment is illustrative of the characteristics of the present invention so as to enable a person skilled in the art to understand the disclosed subject matter and implement the present invention accordingly. The preferred embodiment, however, is not intended to restrict the scope of the present invention. Hence, all equivalent modifications and variations made in the preferred embodiments without departing from the spirit and principle of the present invention should fall within the scope of the appended claims.

The RBD has an amino acid sequence of SEQ ID NO:1, one of the two N-terminal domains has an amino acid sequence of SEQ ID NO:2 or SEQ ID NO:3, and the domain between the RBD and the N-terminal domain has an amino acid sequence of SEQ ID NO:4.

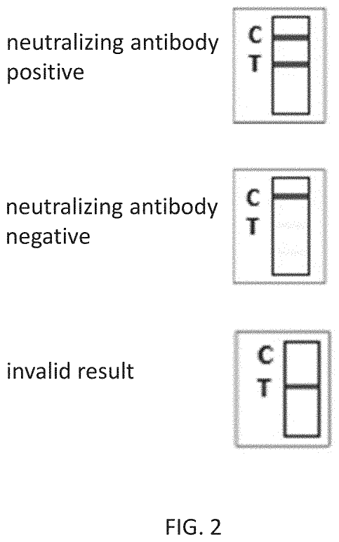

Referring to FIG. 2, if there is neutralizing antibodies against COVID-19 in a specimen, the neutralizing antibodies would first react with the antigen-complex labelled with the color particle and then form an antibody-color particle-antigen-complex molecule flowing on the cellulose membrane 12 via capillary action. When the molecule reaches the test line region (T), a color band appears, it means neutralizing antibody positive result. Still referring to FIG. 2, if there is no neutralizing antibodies against COVID-19 in a specimen, a color band would not appear on the test line region (T), it means neutralizing antibody negative result. Still referring to FIG. 2, the control line region (C) serves as an indicator for validity. Regardless if positive or negative result, the control line region (C) must appear a color band, it also serves as internal control for specimen processing.

Verify that the Present Invention has Quantitative Effect by Viral Neutralization Assay

Antibody inhibition assays make use of the specificity of an antibody to prevent infection (neutralization) or other activity to identify the strain of the infecting agent, usually a virus, or to quantitate antibody responses to a specific strain of virus. The relative antibody concentration is reported as a titer. A titer is the inverse of the greatest dilution, or lowest concentration (e.g., dilution of 1:64=titer of 64), of a patient's serum that retains activity in one of the immunoassays just described. Antibody neutralization of virus inhibits infection and subsequent cytopathologic effects (CPE) in tissue culture cells. In the absence of antibody, the virus infected the monolayer (indicated by CPE). In the presence of the antibody, infection was blocked, preventing CPE (neutralization).

Vero E6 cells were regularly maintained in minimal essential medium (MEM) supplemented with 10% (v/v) fetal bovine serum (FBS). COVID-19 virus was propagated in Vero E6 cells in maintenance medium consisting of MEM supplemented with 2% FBS. For the viral neutralization assay, Vero E6 cells were seeding 2.5×10⁴ cell/well onto 96 well plate and incubated for 18 hours. After incubation, the medium was replacing with 2% MEM to go further test.

COVID-19 positive specimens were collected by Access Biologicals. Access Biologicals was actively collecting convalescent serum from patients that have been diagnosed with COVID-19 using the Roche swab assay. The patients confirmed positive COVID-19 Donors ≥10 and ≤30 days after positive test result.

Serum specimens were inactivated at 56° C. for 30 minutes prior to use and diluted in 1:8. Serial two-fold dilutions of sera were mixed with an equal volume of COVID-19 virus suspension containing 100 folds of median tissue culture infectious dose (TCID50).

The mixture was incubated for 2 hours at 37° C. and then added into Vero E6 cells which containing 2% MEM to incubate for 5 days at 37° C. At post infection day 5, cells were fixed with 10% formaldehyde and stained with 0.1% crystal violet.

Serum neutralization titers were analysed by using Reed-Muench Method to calculate Logarithm 50% end point. The capacities of the neutralizing antibodies are defined as none, poor, moderate and strong neutralization, when the viral neutralization assay titer in blood are tested and resulted in less than 8, 8-20, 21-60, and greater than 60, respectively. Shown as Table 1.

TABLE 1
Results of viral neutralization assay

| | positive (n = 35) | | | negative |
|---|---|---|---|---|
| viral | + | ++ | +++ | − |
| neutralization | (8-20) | (21-60) | (>61) | (<8) |
| assay titer | n = 13 | n = 14 | n = 8 | n = 85 |
| poor | 13 | | | |
| moderate | | 14 | | |
| strong | | | 8 | |
| none | | | | 84 |

| | |
|---|---|
| sensitivity | 100% |
| specificity | 98.8% |

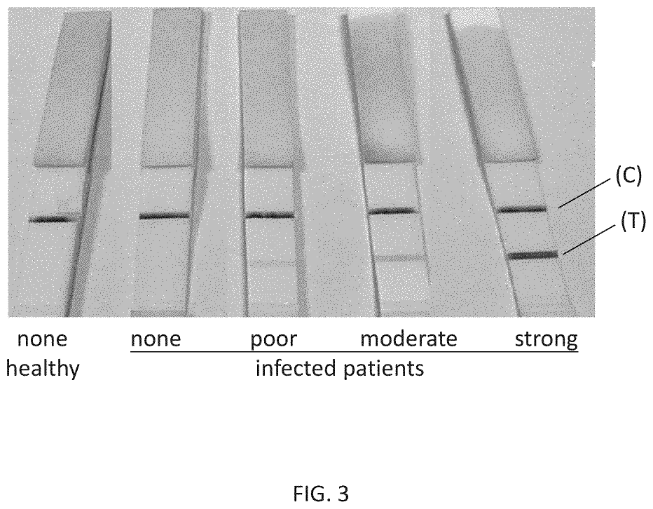

It can be seen from the test data in Table 1 that 35 COVID-19 positive serum specimens were tested without any neutralizing antibody false negative result found, which indicated the sensitivity of the present invention is 100%. 85 COVID-19 positive serum specimens and high-risk groups COVID-19 negative serum specimens were tested with 1 neutralizing antibody false positive result found, which indicated the specificity of the present invention is 98.8%. Please refer to FIG. 3. The present invention provided an actual test result of a representative lateral flow format (strip). From left to right, the specimen in the far left strip came from a healthy individual, therefore the test result was negative and the level of neutralizing antibody against COVID-19 is none. Specimens in other strips came from infected patients, and the test results of detecting the levels of neutralizing antibodies against COVID-19 were in order of none, poor, moderate and strong. The color intensity of the test line region (T) is positive corrected with the antibodies titer from viral neutralization assay. Hence, the present invention is the first medical point-of-care test (POCT) device which can rapidly classify the human blood of COVID-19 infected patients or vaccine recipients into none, poor, moderate and strong neutralizing titer groups.

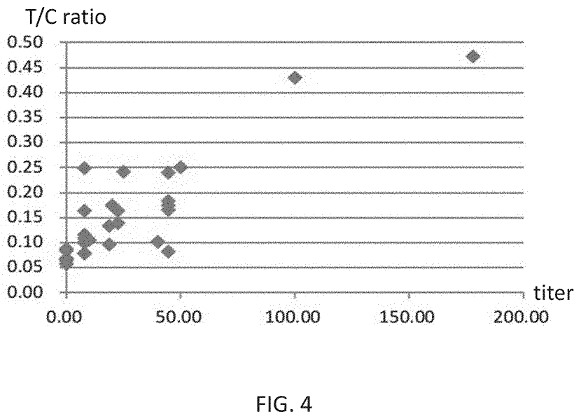

Furthermore, a densitometer was used to scan the greyscale resolution in order to calculate ratio of the test line region (T) and the control line region (C) (T/C ratio). Please refer to FIG. 4. T/C ratio provides a quantitative result for titer assessment of neutralizing antibody against COVID-19. The quantitative result is strong when the ratio is >0.15; the quantitative result is moderate when the ratio is between 0.01 and 0.15; the quantitative result is poor or none when the ratio is <0.01.

Method for Detecting Neutralizing Antibody Against COVID-19

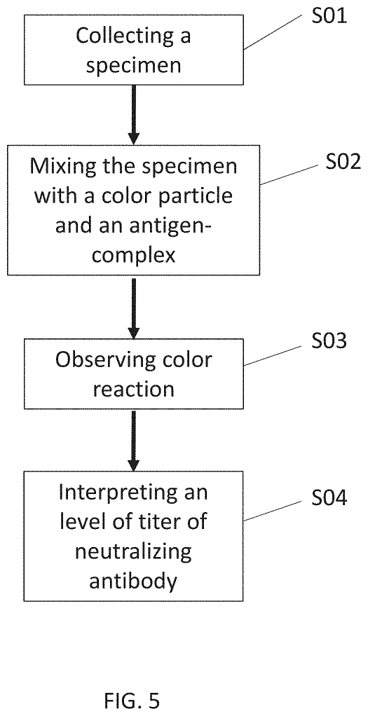

Please refer to FIG. 5. The method for detecting neutralizing antibody against COVID-19 with the usage of the test kit of the present invention comprises: step S01, collecting a specimen from a test subject, the specimen comprises plasma, serum, whole blood, urine and saliva; step S02, mixing the specimen with a color particle and an antigen-complex in the test kit; step S03, observing color reaction of a test result, if there is neutralizing antibodies against COVID-19 in the specimen, color bands appear on the test line region (T) and the control line region (C), it means a neutralizing antibody positive result; if there is no neutralizing antibodies against COVID-19 in a specimen, a color band appears on the control line region (C) but not on the test line region (T), it means a neutralizing antibody negative result; step S04, interpreting an level of titer of neutralizing antibody against COVID-19 in the positive test result, please refer to FIG. 3, titer of neutralizing antibody against COVID-19 is strong when color intensity of the test line region (T) is equal to color intensity of the control line region (C); titer of neutralizing antibody against COVID-19 is moderate when color intensity of the test line region (T) is half of color intensity of the control line region (C); titer of neutralizing antibody against COVID-19 is poor when color intensity of the test line region (T) appears faint color band.

Assay cross-reactivity of the test kit of the present invention was evaluated using serum or plasma specimens which contain antibodies to the pathogens listed in Table 2. No false positivity was found.

TABLE 2
Potentially Cross-Reactivity

| Potential cross reactants | Number of specimens |
|---|---|
| Dengue | 5 |
| HPV | 5 |
| Flu A | 5 |
| Flu B | 5 |
| Tuberculosis | 5 |
| Adenovirus | 5 |
| EBV | 5 |
| RSV | 5 |
| HBV | 5 |
| HCV | 5 |
| chronic obstructive pulmonary disease (COPD) | 5 |
| Marek's disease (MD) | 5 |

What is claimed is:

1. A test kit for detecting neutralizing antibody against COVID-19, said test kit comprising:
an absorbent pat, a color particle and an antigen-complex immobilized at the absorbent pat; and
a cellulose membrane, further comprising a test line region (T) printed with a testing reagent and a control line region (C) printed with a controlling reagent.

2. The test kit of claim 1, wherein titer of neutralizing antibody against COVID-19 is strong when color intensity of the test line region (T) is equal to color intensity of the control line region (C); titer of neutralizing antibody against COVID-19 is moderate when color intensity of the test line region (T) is half of color intensity of the control line region (C); titer of neutralizing antibody against COVID-19 is poor when color intensity of the test line region (T) appears faint color band.

3. The test kit of claim 1, wherein ratio of the test line region (T) and the control line region (C) provides a quantitative result for titer assessment of neutralizing antibody against COVID-19.

4. The test kit of claim 3, wherein the quantitative result is strong when the ratio is greater than 0.15; the quantitative result is moderate when the ratio is between 0.01 and 0.15; the quantitative result is poor or none when the ratio is less than 0.01.

5. The test kit of claim 1, wherein the antigen-complex is a receptor binding domain (RBD) located in COVID-19 spike glycoprotein, two N-terminal domains located in COVID-19 spike glycoprotein, a domain between the RBD and the N-terminal domain, or a combination thereof.

6. The test kit of claim 5, wherein the RBD has an amino acid sequence of SEQ ID NO:1, one of the two N-terminal domains has an amino acid sequence of SEQ ID NO:2 or SEQ ID NO:3, and the domain between the RBD and the N-terminal domain has an amino acid sequence of SEQ ID NO:4.

7. The test kit of claim 1, wherein the color particle comprises a colloid gold, a latex particle, and a silver particle.

8. The test kit of claim 1, wherein the testing reagent is goat anti-human IgG antibody.

9. The test kit of claim 1, wherein the controlling reagent is rabbit anti-RBD antibody.

10. The test kit of claim 1, wherein the test kit is a lateral flow format.

11. A method for detecting neutralizing antibody against COVID-19, said method comprising the steps of:
a. collecting a specimen from a test subject;
b. mixing the specimen with a color particle and an antigen-complex in the test kit as claimed in claim 1;
c. observing color reaction of a test result; and
d. interpreting an level of titer of neutralizing antibody against COVID-19 in a positive test result;
wherein the test kit comprises:
an absorbent pat, the color particle and the antigen-complex immobilized at the absorbent pat; and
a cellulose membrane, further comprising a test line region (T) printed with a testing reagent and a control line region (C) printed with a controlling reagent.

12. The method in claim 11, wherein in the positive test result, titer of neutralizing antibody against COVID-19 is strong when color intensity of the test line region (T) is equal to color intensity of the control line region (C); titer of neutralizing antibody against COVID-19 is moderate when color intensity of the test line region (T) is half of color intensity of the control line region (C); titer of neutralizing antibody against COVID-19 is poor when color intensity of the test line region (T) appears faint color band.

13. The method in claim 11, wherein ratio of the test line region (T) and the control line region (C) provides a quantitative result for titer assessment of neutralizing antibody against COVID-19.

14. The method in claim 13, wherein the quantitative result is strong when the ratio is greater than 0.15; the quantitative result is moderate when the ratio is between 0.01 and 0.15; the quantitative result is poor or none when the ratio is less than 0.01.

15. The method in claim 11, wherein the specimen comprises plasma, serum, whole blood, urine, and saliva.

16. The method in claim 11, wherein the color particle comprises a colloid gold, a latex particle and a silver particle.

17. The method in claim 11, wherein the antigen-complex is a receptor binding domain (RBD) located in COVID-19 spike glycoprotein, two N-terminal domains located in COVID-19 spike glycoprotein, a domain between the RBD and the N-terminal domain, or a combination thereof.

18. The method in claim 17, wherein the RBD has an amino acid sequence of SEQ ID NO:1, one of the two N-terminal domains has an amino acid sequence of SEQ ID NO:2 or SEQ ID NO:3, and the domain between the RBD and the N-terminal domain has an amino acid sequence of SEQ ID NO:4.

19. The method in claim 11, wherein the testing reagent is goat anti-human IgG antibody.

20. The method in claim 11, wherein the controlling reagent is rabbit anti-RBD antibody.

21. The method in claim 11, wherein the test kit is a lateral flow format.


SEQUENCE LISTING (4 sequences)

SEQ ID NO: 1 - protein, 457 aa, SARS-CoV-2 (COVID-19)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFADDDDKAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 2 - protein, 16 aa, SARS-CoV-2 (COVID-19)
LGVYYHKNNKSWMESE

SEQ ID NO: 3 - protein, 15 aa, SARS-CoV-2 (COVID-19)
RSYLTPGDSSSGWTA

SEQ ID NO: 4 - protein, 12 aa, SARS-CoV-2 (COVID-19)
EPVLKGVKLHYT